(19) United States
(12) Patent Application Publication
(10) Pub. No.: US 2009/0148516 A1
(43) Pub. Date: Jun. 11, 2009

(54) TREATED FEED SUPPLEMENT CAPSULE FOR RUMINANTS

(76) Inventor: Thomas C. Jenkins, Central, SC (US)

Correspondence Address: DORITY & MANNING, P.A., POST OFFICE BOX 1449, GREENVILLE, SC, 29602-1449, US

(21) Appl. No.: 11/996,301

(22) PCT Filed: Jul. 24, 2006
(86) PCT No.: PCT/US06/28592

§ 371 Date: Jul. 22, 2008

Related U.S. Application Data
(60) Provisional application No. 60/701,715, filed on Jul. 22, 2005.

(51) Int. Cl. A61K 9/48; A23K 1/16; A23K 1/18

(52) U.S. Cl. 424/456; 426/89; 426/72; 426/61; 426/2

(57) ABSTRACT

A formaldehyde-treated protein capsule for protecting any encapsulated substance from chemical modification in the rumen is generally disclosed. Also, processes for making and using the formaldehyde treated protein capsule arc generally disclosed. The formaldehyde treated capsule can contain substantially no excess formaldehyde on or within the capsule. Also, the protein capsule can be generally treated with formaldehyde after the capsule is loaded with the substance to be protected. The substance to be protected is not limiting by the treatment process. In another embodiment, a method for decreasing the amount of milk fat found in milk provided by a ruminant, such as a dairy cow, is generally provided.

### PRIORITY INFORMATION

The present application claims priority to the provisional application Ser. No. 60/701,715 filed on Jul. 22, 2006, entitled Modified Feed Supplement Capsule for Ruminants.

### BACKGROUND OF THE INVENTION

The assurance of adequate nutrient delivery to the body tissues of ruminant species (such as cattle and sheep) provides benefits not only to the animal but also to consumers of animal products. Specific nutrients are needed in the body tissues of ruminants to maintain optimum health, reproduction, and production of the animal. Nutrients can also be enhanced in animal products and by-products, e.g., meat and milk, to deliver nutraceuticals to humans for improved health and disease resistance. However, due to the nature of the digestive system of ruminants, many of these useful nutrients are often chemically modified by the microbial population of the rumen. For example, microbes in the rumen can convert most of the unsaturated fatty acid consumed by cattle into saturated fatty acid through biohydrogenation, thus depriving the animal tissues of important unsaturated fatty acids needed for normal tissue function. Biohydrogenation also leads to development of animal products, e.g., meat and milk, enriched with less healthy saturated fats.

Many protective coatings have been proposed to protect nutrients from the microbial population of the rumen. The goal of these coatings is to protect the nutrients from chemical modification in the rumen. With suitable protection, the nutrients could be released in a later stage of the digestive process, where they could be absorbed and deposited in body tissues and milk. Unfortunately, many of these protective coatings are limited to use with a small group of nutrients, or even a single nutrient.

One method developed to protect a nutrient from the microbial population of the rumen has been to utilize formaldehyde-treated proteins in some combination with the nutrient. This method has been utilized in an attempt to protect unsaturated fatty acids from biohydrogenation. The formaldehyde-treated protein supplements have been formed by emulsifying unsaturated fatty acids and adding a protein to the emulsion. In the emulsion, the protein forms a barrier between the emulsion solvent and the unsaturated fatty acids. Following formation of the emulsion, formaldehyde can be added to react with the protein.

Unfortunately, these types of formaldehyde-treated protein supplements have several disadvantages. For instance, when the formaldehyde is added to the emulsion containing both the nutrient and protein, the hope is that the formaldehyde will react with only the protein. However, there is no way to prevent the formaldehyde from reacting with the nutrient. Additionally, the creation of a protective coating depends on the formation of the protein-formaldehyde crosslinks within the combined emulsion. Any imperfection in the cross-linked product can significantly reduce the amount of unmodified nutrient that is available for absorption into the blood stream of the ruminant.

In addition, residue formaldehyde can remain within the supplement, due in part to the method of making the formaldehyde-treated protein coatings from the protein/nutrient emulsion. The presence of excess formaldehyde can raise concerns about nutrient overprotection (i.e. inadequate release in the intestines) and also can raise concerns about safety of any food product produced by the ruminant. Moreover, the process of treating the protein/nutrient emulsion with formaldehyde can limit the type of nutrient to be included in the emulsion.

A need exists for a coating that can substantially protect any type of nutrient from chemical modification in the rumen, while still allowing the nutrient to be released and absorbed into the ruminant's bloodstream. Also, a need exists for a coating that does not have a significant amount, if any, unreacted formaldehyde present within the coating. Moreover, a feed supplement that can be formed while avoiding contact between the nutrient to be protected and poisonous reactants, such as formaldehyde, would be of great benefit.

### SUMMARY

Objects and advantages of the invention will be set forth below in the following description, or may be obvious from the description, or may be learned through practice of the invention.

In one embodiment, the present invention is directed to a feed supplement for a ruminant. The feed supplement comprises a nutrient located within a capsule. The capsule comprises a protein treated with an aldehyde, such as formaldehyde. In one particular embodiment, the capsule comprises a gelatin capsule comprising a natural protein derived from plant or animal proteins such as porcine proteins.

The capsule is configured to protect at least about 50% by weight of said nutrient from chemical modification in ruminal fluid after incubation for 24 hours. For example, the capsule can be configured to protect at least about 75% by weight, such as at least about 95%, of said nutrient from chemical modification in ruminal fluid after incubation for 24 hours.

In one embodiment, the nutrient comprises an unsaturated fatty acid, such as an unsaturated fatty acid selected from the group consisting of oleic acid, palmitoleic acid, vaccenic acid, linoleic acid, conjugated linoleic acid, and linolenic acid.

In another embodiment, the invention is directed to a method of protecting nutrients from biohydrogenation in the rumen of a ruminant. The method comprises encapsulating a nutrient within a gelatin capsule. The gelatin capsule comprises a protein. The gelatin capsule is treated with an aldehyde to crosslink the protein of the gelatin capsule in such a manner that the nutrient within the gelatin capsule is substantially prevented from contact with the aldehyde. The treated gelatin capsule can be washed with a solvent. The treated gelatin capsule is washed with a solvent to remove at least about 90% of any unreacted aldehyde.

In yet another embodiment, the present invention is directed to a method of decreasing the percentage of milk fat in milk provided by a ruminant. The method comprises feeding a ruminant a feed supplement. The feed supplement comprises a gelatin protein capsule treated with an aldehyde to crosslink the proteins of the gelatin protein capsule. The treated gelatin capsule encapsulates an unsaturated fatty acid. For example, the gelatin protein capsule can be at least about 95% free of formaldehyde.

For example, the ruminant can be a dairy cow. In some embodiments, the percentage of milk fat in milk collected from the dairy cow can be less than about 85% of the percentage of milk fat in milk collected from the dairy cow prior to feeding the dairy cow the feed supplement, such as less than about 80% of the percentage of milk fat in milk collected from the dairy cow prior to feeding the dairy cow the feed supplement.

Various features and aspects of the present invention are discussed in greater detail below.

### DETAILED DESCRIPTION OF THE PREFERRED EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention covers such modifications and variations as come within the scope of the appended claims and their equivalents.

Generally speaking, the present invention is directed to an aldehyde- treated protein capsule capable of substantially surviving degradation in the rumen of a ruminant and thus subsequently releasing the capsule's contents later in the digestive process. The aldehyde-treated protein capsule can allow the unmodified contents of the capsule to be absorbed by the ruminant following release. In one particular embodiment, the aldehyde-treated protein capsule is a formaldehyde- treated protein capsule.

The aldehyde-treated protein capsule can form or be included in a feed supplement to provide a biologically active substance or nutrient, such as essential unsaturated fatty acids, essential amino acids, pharmaceutical products, enzymes and combinations thereof, to a ruminant. Though generally referred to throughout the following description as a “nutrient” it should be understood that any biologically active material can be protected as herein described from degradation by the microbial population of the rumen. In particular, the aldehyde-treated protein capsule can survive the environment of the rumen with little or no degradation and thus protect the nutrient held inside from degradation due to microbial action. For instance, a lipid can be protected from microbial biohydrogenation. As the digestive process continues and the capsule passes beyond the microorganism population of the rumen, the aldehyde-treated protein capsule can degrade, for instance via acids and/or enzymes of the latter sections of the digestive tract, and the nutrient held inside the coating can then be released and digested by the animal. For example, the aldehyde-treated protein capsule can resist degradation within the rumen such that at least a portion of the nutrient held in the capsule can be released only after reaching the abomasum and/or the intestines. For instance, the aldehyde-treated protein capsule can substantially survive the environment of the abomasum and be broken down by proteinases that are secreted in the first part of the small intestine.

In addition to protecting the nutrient from chemical modification in the rumen, the aldehyde-treated protein capsules can contain substantially no unreacted or residual formaldehyde, and in one embodiment can contain no detectable unreacted or residual formaldehyde, on or within the capsule. In one embodiment, only the outer surface of a capsule comes into contact with an aldehyde during the formation process. Accordingly, the nutrient encapsulated within the capsule can be protected from contact with formaldehyde during the formation process. Moreover, following formation of the capsule, the capsule can include little or no unreacted formaldehyde either at the surface or within the protective coating of the capsule. As such, the capsule can be free of residual formaldehyde resulting in reduced health concerns in administering the capsule. For example, in one embodiment, the capsule can be at least about 90% free of unreacted formaldehyde, such as at least about 95% free. In some particular embodiments, the capsule can be at least about 99% free of unreacted formaldehyde, such as having no detectable amounts of unreacted formaldehyde.

The aldehyde-treated protein capsule coating of the capsule can include any protein that can be cross-linked with an aldehyde cross-linking agent. Such proteins are well known in the art and as such, a detailed listing is not contained herein. For example, the capsule can include natural proteins including animal proteins or plant proteins. For instance, a gelatin capsule can be formed including a natural animal protein, such as from pigs, cows, horses, and the like.

In one embodiment, protein capsules of porcine or plant origin that comply with current U.S. regulations concerning the inclusion of proteins in cattle feed, can be utilized. Such capsules are known in the art and can be formed from suitable proteins available on the wholesale or retail market. For instance, in one particular embodiment, the protein capsule can be a gelatin protein capsule formed from porcine protein supplied by Capsugel, Inc., of Greenwood, S.C. Formation of the protein capsules can be performed through any suitable process. In one embodiment, the desired nutrients can be encapsulated within the capsule during the formation process.

Following formation and encapsulation of the nutrient, the protein capsule can be treated with an aldehyde, such as formaldehyde, to enable the treated protein capsule to survive the microbial environment of the rumen with little or no degradation. In one preferred embodiment, only the outer surface of the protein capsule can be treated with formaldehyde. As the nutrient to be protected can be encapsulated within the formed protein capsule prior to contact of the capsule with formaldehyde, no significant amount, if any, of the formaldehyde need contact the substance during the treatment process. Moreover, unreacted aldehyde can be removed following the cross-linking reaction. As such, little or no residual formaldehyde need remain in or on the protein capsule.

The aldehyde can crosslink the protein of the capsule to create a microbial resistant aldehyde-treated protein capsule. In one embodiment, the cross-linking agent can be formaldehyde. According to this embodiment, formaldehyde can crosslink proteins in the capsule through formation of methylene bridges between reactive groups of the protein. Without wishing to be bound by theory, it is believed that the crosslinked proteins of the aldehyde-treated protein capsule can inhibit degradation of the protein that would normally occur in the microbe-rich environment of the rumen without crosslinking. Thus, the capsule and its contents can substantially survive the rumen environment with little or no degradation.

In one embodiment, the protein capsule can protect at least about 50% of the encapsulated nutrients from chemical modification in the rumen, such as at least about 75%. In other embodiments, at least about 85% of the encapsulated substances can be protected from chemical modification in the rumen, such as greater than about 90%. In one embodiment, the capsule can protect 100% of the encapsulated substances from chemical modification in the rumen.

In general, the formed capsules can be any size or shape capable of being ingested by a ruminant. For instance, the protein capsules can have varying shapes from round to elongated, can have varying sizes, and can vary in thicknesses of the cross-linked protein layer.

The disclosed capsules can be combined with other materials and be ingested by the animals in this form. For example, the capsules of the invention can be mixed or otherwise combined with other materials commonly fed to the animals, such as processed dry feed materials or salt, and can be ingested by the animal in this combination form.

Processes of Protecting Nutrients

The present disclosure is also generally directed toward processes for making an aldehyde-treated protein capsule that is capable of protecting nutrients from chemical modification in the rumen. Moreover, the formation methods can also prevent ingestion of an unreacted cross-linking agent by the ruminant. The process generally involves the steps of encapsulating a nutrient within a protein capsule and then treating the formed protein capsule, particularly on the outer surface of the capsule, with an aldehyde. For example, one or more nutrients and/or other bioactive materials can first be encapsulated by a protein coating and thus protected from significant contact with the aldehyde solution during later aldehyde treatment. In one embodiment, the nutrients can be completely protected from any contact with the aldehyde solution during both formation of the capsule and treatment of the capsule.

When formaldehyde is used, the treating solution can have any amount of formaldehyde suitable to react with the amount of protein forming the capsule. For instance, the formed capsules can be contacted with a solution including at least about 0.1 wt % formaldehyde by weight of the solution. For example, the solution can include from about 0.1 wt % to about 37 wt % formaldehyde. For instance, in some embodiments, the formaldehyde solution can have from about 1 wt % to about 20 wt % formaldehyde, such as about 3 wt % to about 6 wt %. In one embodiment, the treatment solution can have about 5 wt % formaldehyde.

In other embodiments, the treating solution can contain aldehydes, other than formaldehyde, including, but not limited to, acetaldehyde, glutaraldehyde, and any similar series of common aldehydic solution.

Any method of treating the protein capsules with the formaldehyde solution can be utilized. For instance, the untreated protein capsules encapsulating the nutrient can be added to the treatment solution containing the aldehyde. Then, the solution containing the capsules can be stirred or otherwise agitated. If desired, the mixture containing the capsules and formaldehyde can be heated.

After treatment, the capsules can be collected and separated from the aldehyde solution for instance, by straining the mixture. Optionally, the treated capsules can then be dried, either in ambient air or by adding heat. Drying the capsules, especially with heat, can remove most of any residual or unreacted aldehyde from the capsule.

The treated capsules can be washed to remove any remaining aldehyde from the surface of the capsule. Any suitable solvent can be utilized to wash the treated capsule, such as water, ethanol, methanol, propanol, ethyl acetate, ethylene glycol, propylene glycol, and the like. Washing can be performed by any method, such as rinsing the capsule with the solvent or submersing the capsules into the solvent (followed by separation of the solvent from the capsules).

After washing, the capsules can be dried, by ambient air or utilizing heat as desired, to provide a treated protein capsule that has no significant amount, if any, aldehyde in or on the capsule.

In another embodiment, the protein capsule shells can be formed and treated with aldehyde prior to encapsulation of the nutrients. In this embodiment, the protein capsule shell can be washed to remove any residual formaldehyde and dried. Then, the treated protein capsule shells can be loaded with the material to be protected.

The processes of the present disclosure can advantageously be utilized in a large-scale process to treat a large amount of protein capsules. As such, the disclosed processes are well suited for production of mass quantities of treated capsules that can protect any nutrient from chemical modification in the rumen.

Biohydrogenation in a Ruminant

Ruminants are a class of animals, such as cows, goats, deer, moose and sheep, distinguished by their multi-compartment stomachs. Typically, ruminants have 4 major stomach compartments: the rumen, the reticulum, the omasum, and the abomasum. During the digestive process, feed passes first to the rumen, the largest compartment of the digestive system. The rumen of mature cattle describes an aqueous environment of roughly a 40-50 gallon capacity that supports a microbial population including bacteria, fungi, and protists at a relatively constant temperature and pH. The microbial population of the rumen is responsible for fermentation and transformation of dietary fiber that enables the animal to survive and thrive on fodder that is indigestible by other animals.

Among other functions, the microbial population of the rumen is responsible for lipolysis and biohydrogenation of the lipid found in the animal's feed including hay, silage, grain, and pasture. One waste product of the biohydrogenation process carried out by the microbial population in the rumen is saturated fatty acid (SFA). For instance, unsaturated fatty acids (UFA), including both monounsaturated and polyunsaturated fatty acids, either existing in the feed as such or formed during the initial digestive processes by the microbes, can be converted into saturated fatty acid during biohydrogenation.

Linoleic acid is a common UFA found in animal feed. Biohydrogenation of linoleic acid in the rumen can begin with the conversion of linoleic acid to conjugated linoleic acid (CLA), in which the total number of double bonds on the backbone of the carbon chain remains the same but one of the double bonds is shifted to a new position by microbial enzymes. Many types of CLA are produced in the rumen of dairy cows, but a common CLA produced from biohydrogenation of linoleic acid is cis-9, trans-11 C18:2. As the biohydrogenation progresses, double bonds in the CLA intermediates can then be hydrogenated further to trans fatty acids having only one double bond. A final hydrogenation step by the ruminal microbes can eliminate the last double bond yielding the SFA stearic acid as the final end product. Waste products of the microbial processing such as stearic acid can be absorbed by the animal through the lining of the rumen wall or can be passed to the rest of the digestive system with the remaining feed that is not subject to biohydrogenation.

Biohydrogenation of lipids in the rumen can greatly reduce the quantity of dietary UFA available for uptake into the bloodstream during digestion. For example, intake of linoleic acid by dairy cows typically ranges from about 70 to about 200 g/day, but only about 10 to about 50 grams of this ingested linoleic acid usually survives biohydrogenation to reach the small intestine. In contrast, about 500 g of saturated stearic acid can reach the small intestine of a dairy cow each day, even though only a few grams of stearic acid are consumed. Typically, stearic acid can be the primary fatty acid absorbed by cows regardless of the quantity of UFA consumed in the diet.

After processing in the rumen, biohydrogenation products pass through the omasum and the abomasum, and into the intestines, which is the primary site of absorption. The fermentation products of rumen digestion as well as the remaining biohydrogenation products of microbial digestion can be absorbed into the blood stream of the animal or excreted as waste. Importantly, however, conversion of UFA to SFA will primarily only occur during the biohydrogenation processes of the rumen, and UFA that survives digestion by the microbial population of the rumen can be absorbed as such, as the additional enzymatic digestion processes of the remaining digestive system will generally not convert UFA to SFA.

Nutrients Protected by the Formaldehyde-treated Protein Capsule

While the methods and products disclosed herein can be greatly beneficial in preventing the biohydrogenation of lipids described above, the capsules are not limited to protecting such nutrients. In fact, the disclosed capsules can be loaded with any nutrient or other substance for protection from degradation by rumen microorganisms. For example, a nonlimiting list of nutrients that can be protected by the capsule of the present disclosure includes fatty acids (such as any of the omega-3 and omega-6 classes of unsaturated fatty acids, conjugated fatty acids such as cis-9, trans-11 CLA), amino acids (such as lysine and methionine), and vitamins (such as any of the B-vitamins and choline). Other bioactive substances can be protected, including, but not limited to, pharmaceuticals (such as drugs), biologics, enzymes (such as digestive enzymes with activity in the intestines), and absorbable peptide sequences having tissue effects.

In one embodiment, the nutrients to be protected can be unsaturated fatty acids (UFA). For instance, UFA can be directly encapsulated within the protein coating prior to treating the protein capsule with the aldehyde. As such, the UFA can be substantially protected from contact with the aldehyde, preventing any contact or reaction between the aldehyde and the encapsulated material. Thus, the presence of any unreacted or residual aldehyde, e.g., formaldehyde can be prevented within the capsule.

In one embodiment, a long chain fatty acid up to about 30 carbons in length and including anywhere from 1 to about 6 double bonds, can be held in the protein capsules. For example, the unsaturated fatty acid can be from about 10 carbons to about 26 carbons in length. The unsaturated fatty acids can include any of the omega 3 fatty acids or any of the omega 6 fatty acids and can also include trans-fatty acids, such as found in conjugated linoleic acids. For instance, unsaturated fatty acids including, but not limited to, oleic acid (C18:1), palmitoleic acid (C16:1), vaccenic acid (C18:1), linoleic acid (C18:2), conjugated linoleic acid (CLA), linolenic acid (C18:3), arachidonic acid (C20:4), eicosapentaenoic acid (C20:5), and/or docosahexaenoic acid (C22:6) can be encapsulated within the protein capsules either individually or in combination.

In one embodiment, more complex materials can be encapsulated within the protein capsule and protected from microbial digestion according to the present invention. For example, in one embodiment, complex fats such as esterfied lipids (triacylglycerols, phospholipids, and the like) could be encapsulated in the protein capsule. Following suitable degradation of the coatings, the complex fats can be released from the capsule and subjected to enzymatic digestion to form UFA that can then be absorbed by the animal.

In general, the disclosed capsules can be utilized to deliver up to about 2 pounds of encapsulated nutrient to the digestive tract of an animal per day. In one embodiment, an animal can be fed the disclosed capsules so as to deliver between about one and about two pounds of nutrients to the animal. In another embodiment, an animal can be fed less than about one pound per day of a nutrient via the disclosed capsules. For example, an animal can be fed less than about 200 grams of a nutrient per day via the disclosed capsules, or less in other embodiments, such as less than about 100 grams per day, less than about 50 grams per day, less than about 10 grams per day, or less than about 5 grams per day.

Process for Decreasing the Amount of Milk Fat

In another embodiment, the aldehyde-treated protein capsules can be utilized to decrease the amount of milk fat present in the milk of a ruminant. For example, a the milk collected from a ruminant, such as a dairy cow, can have less milk fat after being fed unsaturated fatty acids encapsulated by the treated protein capsule described above. According to this embodiment, more of the unsaturated fatty acid can survive biohydrogenation in the rumen to be absorbed into the bloodstream of the ruminant. Thus, the milk produced from the ruminant can have more unsaturated fatty acid content, while having a decreased amount of saturated fatty acid content (milk fat) as compared to a cow fed the same amounf of UFA in an unprotected form.

### EXAMPLES

### Example 1

### Preparation of an Exemplary Formaldehyde Treated Capsule

Gelatin capsules loaded with conjugated linoleic acid (CLA) were purchased from Capsugel, Inc., of Greenwood, S.C. Each capsule was loaded with 59+/−1 mg of fatty acids, containing about 12.3% oleic acid and about 74.2% total CLA, which is the sum of (A) about 36% cis-9, trans-11 -CLA, (B) about 35.7% trans-10, cis-12-CLA; and (C) about 2.5% trans-9, trans-11-CLA.

The gelatin capsules were immersed in a formaldehyde solution diluted to 5% and agitated for about 15 minutes. Then, the capsules were strained and allowed to air dry for about 10 minutes. Next, the capsules were dried at 37° C. for about 30 minutes. The dried capsules were rinsed in 75% ethanol (ethyl alcohol, EtOH) for about 15 minutes. After straining, the capsules were allowed to dry at ambient temperatures for about 15.5 hours.

### Example 2

### Comparison

Protection was assessed by placing treated capsules, prepared according to Example 1 above, in nylon bags containing ruminal fluid comprising cultures of mixed rumen microorganisms. For comparison, untreated capsules containing the same amounts of fatty acids were also placed in nylon bags containing cultures of mixed rumen microorganisms. The capsules were incubated for 24 hours in a Daisy Incubator sold by ANKOM Technologies, Inc. of Macedon, N.Y.

After incubation, the samples were removed, freeze-dried, weighed, and analyzed for fatty acid content. The treated capsules were intact with an average weight loss of 4.0% +/−2.3%. In contrast, the untreated capsules were not visible and could not be recovered from the incubation mixture, presumably due to their complete degradation by the microbial population and total release of contents.

The treated capsules contained similar oleic acid concentrations of about 12.4% and total CLA concentrations of about 69.7%, as before incubation. The content was analyzed by gas chromatography (GC) with fatty acid methyl esters prepared by reaction in sodium methoxide followed by methanolic HCl for 10 minutes each.

Table 1 shows the results of the treated capsules before and after incubation, in mg (error is +/−0.05):

TABLE 1

| Treated capsule: | 0 hour | 24 hour | % Retention |
|---|---|---|---|
| capsule weight | 603.8 | 579.9 | 96 |
| total FA | 44.0 | 44.2 | 101 |
| total oleic acid | 5.4 | 5.4 | 100 |
| total CLA | 32.8 | 30.4 | 93 |

### Example 3

### Cow Study

Four sets of Multiparous Holstein cows (n=16, 185+/−19 DIM), used in a randomized complete block design based on DIM and milk production, were fed CLA for 18 days, after a pre-treatment of 7 days with no additives. The post-treatment was 11 days of respective treatments added. The four sets of cows were fed the following:

A: 100 untreated protein capsules containing 20.4 g of CLA per day.

B: 100 treated protein capsules, such as described above in Example 1, containing 20.4 g of CLA per day.

C: 250 grams of calcium salts of CLA per day (supplied from NutriScience Technologies, Inc. of Fairlawn, Ohio).

D: no CLA (control group).

Data were averaged over the last 3 days of each period. The statistical analysis was determined as a randomized complete block design using a standard analysis of variance method. And, a comparison of diet effects was conducted using LSD. The resulting milk fat percentage is shown in table 2:

TABLE 2
Percentages of milk fat (error of +/−0.05)

| Period | A | B | C | D |
|---|---|---|---|---|
| Pre-treatment | 3.64 | 3.42 | 3.18 | 3.48 |
| Post-treatment | 3.41 | 2.71 | 2.17 | 3.55 |
| % of Original | 93.68 | 79.24 | 85.22 | 102 |
| Milk Fat | | | | |

As the results show, milk fat depression occurred from the cows fed treated, but not untreated, capsules. Also, the greatest percentage of milk fat depression occurred with the treated capsules. Thus, the percentage of milk fat of milk collected from the dairy cow can be less than about 85% of the percentage of milk fat of milk collected from the dairy cow prior to feeding the dairy cow the feed supplement, such as less than about 80%.

What is claimed:

1. A feed supplement for a ruminant comprising
a capsule comprising a protein cross-linked with an aldehyde; and
a nutrient located within said capsule, wherein said capsule is configured to protect at least about 50% by weight of said nutrient from chemical modification in ruminal fluid after incubation for 24 hours.

2. A feed supplement as in claim 1, wherein said aldehyde comprises formaldehyde.

3. A feed supplement as in claim 1, wherein said capsule comprises a gelatin capsule comprising a natural protein derived from plant proteins or porcine proteins.

4. A feed supplement as in claim 1, wherein said capsule is configured to protect at least about 75% by weight of said nutrient from chemical modification in ruminal fluid after incubation for 24 hours.

5. A feed supplement as in claim 1, wherein said capsule is configured to protect at least about 95% by weight of said nutrient from chemical modification in ruminal fluid after incubation for 24 hours.

6. A feed supplement as in claim 1, wherein said nutrient comprises an unsaturated fatty acid.

7. A feed supplement as in claim 6, wherein said unsaturated fatty acid is selected from the group consisting of oleic acid, palmitoleic acid, vaccenic acid, linoleic acid, conjugated linoleic acid, linolenic acid.

8. A feed supplement as in claim 1, wherein said nutrient is selected from the group consisting of amino acids, vitamins, pharmaceuticals, biologics, enzymes, peptides, and mixtures thereof.

9. A method of protecting nutrients from modification in the rumen of a ruminant, the method comprising
encapsulating a nutrient within a gelatin capsule, wherein said gelatin capsule comprises a protein;
contacting the gelatin capsule with an aldehyde cross-linking agent, wherein the nutrient within the gelatin capsule is substantially prevented from contact with the aldehyde;
washing the treated gelatin capsule with a solvent; and
drying the treated gelatin capsule to remove at least about 90% of any unreacted aldehyde.

10. A method as in claim 9, wherein said aldehyde comprises formaldehyde.

11. A method as in claim 10, wherein the gelatin capsule is contacted with formaldehyde that is in a solution including between about 0.1% and about 37% by weight formaldehyde.

12. A method as in claim 9, wherein said nutrient comprises an unsaturated fatty acid.

13. A method as in claim 9, wherein said gelatin capsule comprises a natural protein derived from plant proteins or animal proteins.

14. A method as in claim 13, wherein said protein is a natural porcine protein.

15. A method as in claim 9, wherein said nutrient is selected from the group consisting of amino acids, vitamins, pharmaceuticals, biologics, enzymes, peptides, and mixtures thereof.

16. A method of decreasing the percentage of milk fat in milk provided by a ruminant, the method comprising
feeding a ruminant a feed supplement, wherein the feed supplement comprises a gelatin protein capsule including an aldehyde cross-linked protein, wherein the treated gelatin capsule encapsulates an unsaturated fatty acid.

17. A method as in claim 16, wherein the aldehyde comprises formaldehyde.

18. A method as in claim 16, wherein the gelatin protein capsule is at least about 95% free of formaldehyde.

19. A method as in claim 16, wherein the unsaturated fatty acid is selected from the group consisting of oleic acid, palmitoleic acid, vaccenic acid, linoleic acid, conjugated linoleic acid, linolenic acid.

20. A method as in claim 16, wherein the ruminant is a dairy cow.

21. A method as in claim 20, wherein the percentage of milk fat of milk collected from the dairy cow is less than about 85% of the percentage of milk fat of milk collected from the dairy cow prior to feeding the dairy cow the feed supplement.

22. A method as in claim 13, wherein said gelatin capsule comprises a natural protein derived from plant proteins or porcine proteins.
